(19) United States
(12) Patent Application Publication
(10) Pub. No.: US 2026/0216188 A1
(43) Pub. Date: Jul. 30, 2026

(54) SILDENAFIL PHARMACEUTICAL FORMULATIONS

(71) Applicant: FERRING BV, Hoofddorp (NL)

(72) Inventors: Parag LOKHANDE, Thane (IN); Ankit BAHETI, Indore (IN)

(73) Assignee: FERRING BV, Hoofddorp (NL)

(21) Appl. No.: 19/148,598

(22) PCT Filed: Jan. 11, 2024
(86) PCT No.: PCT/EP2024/050561

§ 371 Date: Jul. 16, 2025

(30) Foreign Application Priority Data
Jan. 17, 2023 (IN) 202311003359

(51) Int. Cl. A61K 31/519; A61J 3/02; A61K 9/00; A61K 9/19

(52) U.S. Cl. CPC A61K 31/519; A61J 3/02; A61K 9/0056; A61K 9/19

(57) ABSTRACT

A solid pharmaceutical formulation comprising a mixture of (1) lyophilized granules comprising sildenafil or a pharmaceutically acceptable salt thereof as active ingredient and (2) a non-lyophilized composition, wherein the formulation comprises 35% to 50% by weight of the lyophilized granules (1) and dissolves in the oral cavity and the non-lyophilized composition (2) is a solid blend composition comprising flow enhancing agents, a package containing the formulation, and a process for the manufacture of the formulation.

### FIELD OF THE INVENTION

The present invention relates to solid pharmaceutical formulations of sildenafil or a pharmaceutically acceptable salt thereof, to methods of making them and to solid blend compositions for use in the manufacture of the pharmaceutical formulations.

### BACKGROUND OF THE INVENTION

Pharmaceutical dosage forms which rapidly dissolve when placed in the mouth of the patient, thereby delivering the desired dose of the pharmaceutically active substances, are particularly useful when the dosage form can be taken without water. Such dosage forms are generally preferred by people with difficulties in swallowing and/or taking tablets or capsules.

Rapidly dissolving dosage forms are generally prepared using a multistep process. Some of these processes use a lyophilization step during which solvent is removed from a solution and/or suspension comprising a pharmaceutically active substance, carrier material and solvent.

Pharmaceutical formulations manufactured by lyophilization have various advantages but also various challenges such as issues of free flow, moisture absorption, bitter taste, dose uniformity, content uniformity and pharmacokinetics. The present invention aims at overcoming these challenges.

### SUMMARY OF THE INVENTION

The present invention provides a novel blend composition of sorbitol and liquid paraffin for use in the improvement of formulation properties of pharmaceutical formulations in several parameters, i.e. flow properties, moisture protection, antistatic properties and compressibility (Carr Index). The blend composition also contains tartaric acid and/or citric acid, in particular for improving dissolution characteristics of pharmaceutical formulations containing sildenafil.

The present invention thus provides a solid pharmaceutical formulation comprising a mixture of (1) lyophilized granules comprising sildenafil or a pharmaceutically acceptable salt thereof as active ingredient and optionally a base, and (2) a non-lyophilized composition that is a solid blend composition comprising flow enhancing agents.

The present invention further provides a solid blend composition, comprising or consisting of sorbitol, liquid paraffin and optionally tartaric acid and/or citric acid, for use in the manufacture of the pharmaceutical formulation of the invention.

The present invention also provides a process for the manufacture of a solid pharmaceutical formulation of the invention comprising:
(a) forming lyophilized granules (1) by subliming the solvent from a preparation comprising sildenafil or a pharmaceutically acceptable salt thereof and a solvent;
(b) forming a solid blend composition (2) comprising or consisting of sorbitol, liquid paraffin and an organic acid selected from one or both of tartaric acid and citric acid; and
(c) mixing the solid blend composition (2) obtained in step (b) with the lyophilized granules (1) obtained in step (a).

### FIGURES

FIG. 1 compares the dissolution rates in water for the formulations of Examples 1 to 4 and the commercial product Viagra® 100 mg tablet.

FIG. 2 compares the dissolution rates in water for the formulations of Comparative Examples A to D and the commercial product Viagra® 100 mg tablet.

FIG. 3 is a photograph of a stick pack of the invention.

### DETAILED DESCRIPTION OF THE INVENTION

The pharmaceutical formulation of the present invention dissolves in the oral cavity and comprises 35% to 50% by weight of the lyophilized granules (1) together with the non-lyophilized composition (2) that is a solid blend composition comprising flow enhancing agents.

In one embodiment, the formulation provides at least one of the following pharmacokinetic parameters upon oral administration to a human subject: AUC (0-10 min) of at least 3.5 ng·h/mL; and AUC (0-30 min) of at least 150 ng·h/mL. The AUC value is determined as further set out in the experimental section.

The flow enhancing agent in the solid blend composition (2) are preferably represented by a combination of sorbitol and liquid paraffin.

In one embodiment, the pharmaceutical formulation of the present invention comprises 0.01% to 30% by weight of the active ingredient, 35% to 50% by weight of the lyophilized granules (1), 50% to 65% by weight of the solid blend composition (2), and 0% to 15% by weight of one or more pharmaceutically acceptable excipients as further constituent components of the formulation, and the solid blend composition (2) comprises 85% to 93% by weight of sorbitol, 5% to 14.9% by weight of an organic acid selected from one or both of tartaric acid and citric acid, and 0.1% to 5% by weight of liquid paraffin. The solid blend composition (2) preferably comprises 90.5% to 92.0% by weight of sorbitol, 5.0% to 9.0% by weight of an organic acid selected from one or both of tartaric acid and citric acid, and 0.5% to 3.5% by weight of liquid paraffin.

The formulation preferably comprises 10% to 20% by weight of the active ingredient, 37.5% to 42.5% by weight of the lyophilized granules (1), 57.5% to 62.5% by weight of the solid blend composition (2), and 0.0% to 5.0% by weight of one or more pharmaceutically acceptable excipients as further constituent components of the formulation.

In one embodiment, the formulation consists of 37.5% to 42.5% by weight of the lyophilized granules (1) and 57.5% to 62.5% by weight of the solid blend composition (2) and the solid blend composition (2) consists of 90.5% to 92.0% by weight of sorbitol, 5.0% to 9.0% by weight of an organic acid selected from one or both of tartaric acid and citric acid, and 0.5% to 3.5% by weight of liquid paraffin.

In one embodiment, the active ingredient of a formulation of the invention is sildenafil citrate.

In one embodiment, the organic acid is tartaric acid.

In one embodiment, the solid pharmaceutical formulation is a free-flowing formulation.

The lyophilized granules (1) comprise sildenafil or a pharmaceutically acceptable salt thereof and may optionally comprise further pharmaceutically acceptable excipients, in particular those that are conventionally used in a process of producing lyophilized granules. The pharmaceutically acceptable excipients can be selected from sugars, sugar alcohols, monosaccharides, disaccharides, trisaccharides, polysaccharides, proteins, amino acids, gums and the like, which include without limitation, mannitol, trehalose, raffinose, inositol, pullulan, sucrose, lactose, dextrose, erythritol, xylitol, lactitol, maltitol, isomalt, alanine, arginine, threonine, glycine, cysteine, serine, histidine, valine, proline, lysine, asparagine, glutamine, ribose, glucose, galactose, fructose, maltose, maltotriose, guar gum, xanthan gum, tragacanth gum, veegum and so forth. Generally, the lyophilized granules (1) do not comprise paraffin. In one embodiment of the invention, the lyophilized granules (1) further comprise a base such as one or more selected from sodium hydroxide, potassium hydroxide, magnesium hydroxide, aluminum hydroxide and calcium hydroxide, preferably sodium hydroxide.

In one embodiment of the invention, the lyophilized granules (1) comprise 35% to 46% by weight of sildenafil or a pharmaceutically acceptable salt thereof (preferably sildenafil citrate), 39% to 65% by weight of sugar alcohol (preferably mannitol), and 0% to 15% by weight of base (preferably sodium hydroxide). More preferably, the lyophilized granules (1) comprise 35% to 44% by weight of sildenafil or a pharmaceutically acceptable salt thereof (preferably sildenafil citrate), 46% to 60% by weight of sugar alcohol (preferably mannitol), and 5% to 10% by weight of base (preferably sodium hydroxide). The proportion of the base is preferably chosen such that the weight ratio of organic acid in the solid blend composition (2) to the base in the lyophilized granules (1) is at least 1.6:1, more preferably at least 2:1. The weight ratio of organic acid to base has no particular upper limit. It may be up to 10:1 or up to 5:1.

The solid blend composition (2) preferably comprises 85% to 93% by weight of sorbitol, 5% to 14.9% by weight of an organic acid selected from one or both of tartaric acid and citric acid, and 0.1% to 5% by weight of liquid paraffin. The solid blend composition (2) more preferably comprises 90.5% to 92.0% by weight of sorbitol, 5.0% to 9.0% by weight of an organic acid selected from one or both of tartaric acid and citric acid, and 0.5% to 3.5% by weight of liquid paraffin. Generally, the solid blend composition (2) does not comprise active ingredient.

In addition to the components (1) and (2) discussed above, the formulation may also include other excipients (auxiliary agents, accessory agents) such as, but not limited to fillers, thickeners (including but not limited to guar gum and xanthan gum), binders, diluents, lubricants, pH adjusting agents, protecting agents, viscosity enhancers, wicking agents, non-effervescent disintegrants, effervescent disintegrants, surfactants, anti-oxidants, wetting agents, colorants, flavoring agents, taste-masking agents, sweeteners, preservatives and so forth.

In an embodiment of the invention, the formulation is obtained by the process of the invention including steps of forming the solid blend composition (2) by blending sorbitol, liquid paraffin and an organic acid selected from one or both of tartaric acid and citric acid, and adding the solid blend composition (2) to the active ingredient, which is in the form of lyophilized granules (1).

The present invention further provides a novel solid blend composition comprising or consisting of sorbitol, liquid paraffin and an organic acid selected from one or both of tartaric acid and citric acid for use in the manufacture of a pharmaceutical formulation comprising sildenafil or a pharmaceutically acceptable salt thereof as active ingredient. The solid blend composition preferably comprises 85% to 93% by weight of sorbitol, 0.1% to 5% by weight of liquid paraffin, and 5% to 14.9% by weight of an organic acid selected from tartaric acid and citric acid. More preferably, the solid blend composition comprises 90.5% to 92.0% by weight of sorbitol, 0.5% to 3.5% by weight of liquid paraffin, and 5.0% to 9.0% by weight of an organic acid selected from one or both of tartaric acid and citric acid. In one embodiment, the blend composition consists of the indicated components, in the indicated preferred and more preferred proportions.

The present invention also provides a process for the manufacture of a solid pharmaceutical formulation of the invention comprising:
(a) forming a lyophilized granules (1) by subliming the solvent from a preparation comprising sildenafil or a pharmaceutically acceptable salt thereof and a solvent and optionally a base;
(b) forming a solid blend composition (2) comprising or consisting of sorbitol, liquid paraffin and an organic acid selected from one or both of tartaric acid and citric acid;
(c) mixing the solid blend composition (2) obtained in step (b) with the lyophilized granules (1) obtained in step (a).

The blend composition (2) of sorbitol and liquid paraffin is mixed with lyophilized granules (1) by methods known in the art such as geometric mixing or mixing with any type of blender.

The process of the invention allows obtaining a solid pharmaceutical formulation in the form of a free-flowing granules, which is a preferred embodiment of the present invention.

Preferred proportions of the active ingredient, sorbitol, liquid paraffin, organic acid and solid blend composition and preferred types and proportions of optional further pharmaceutically acceptable excipients, in particular base, in the process of the present invention are the same as in the pharmaceutical formulation of the present invention.

The solvent used in the preparation to be lyophilized, comprising active ingredient and optionally further pharmaceutically acceptable excipients such as sugar alcohols and base, typically is water.

The terms “active ingredient” and “pharmaceutically active ingredient” are used interchangeably herein.

A suitable route of administration for a dosage form of the present invention is oral administration.

In one embodiment, a pharmaceutical formulation of the invention can be administered to a patient in the form of granules.

In another aspect, a pharmaceutical formulation of the invention is contained in a package dispensing single doses of the formulation, also named a unit dosage form. Non-limiting examples of a package/unit dosage form are a sachet and a stick pack.

In another embodiment, a pharmaceutical formulation of the invention, e.g., in the form of granules, is packed in a multi-dosage form, i.e. a dosage form allowing withdrawing multiple doses of arbitrary size. Non-limiting examples of a multi-dosage form are a bottle and any other container capable of holding granules.

A patient administered with a free-flowing granule formulation of the invention will be certain that he receives the entire dosage, i.e. there will be no residue left in the package/unit dosage form.

In one embodiment, the oral administration is carried out by emptying a sachet, stick pack or (any other) unit pack containing the granules onto the tongue and swallowing the content.

In another embodiment, the oral administration is carried out by dissolving the granules in a solvent (e.g. a glass of water) and then drinking the solution.

The exact dose and regimen of administration of the dosage form will necessarily depend upon the therapeutic effect to be achieved and may vary with the route of administration, and the age and condition of the individual subject to whom the formulation is to be administered. At times patients may be instructed to take two or any other number of unit dosage forms, e.g. stick packs, in a single administration or at times only a portion, such as half or a quarter, of the unit dosage form in a single administration.

The formulations of the invention are unique in that they have improved flow properties, moisture content, antistatic properties and compressibility and exhibit improved pharmacokinetic properties upon administration.

### Examples

The invention is further described in the following examples, which are not in any way intended to limit the scope of the inventions as claimed.

Materials and methods

| Material | Manufactured by | Supplied by |
|---|---|---|
| Citric acid | Merck | Merck |
| Liquid paraffin | Merck | Merck |
| Sorbitol | Roquette | Signet India |
| Sodium Hydroxide | Merck | Merck |
| Mannitol | Roquette France | Signet India |
| Hydroxypropyl cellulose L | Ashland | Ashland |
| Tartaric acid pellets | Ideal cures | Ideal cures |
| Sildenafil citrate | Rakshit drugs | Rakshit drugs |

Example 1

| | |
|---|---|
| | Quantity per 1000 |
| Ingredients | Stick Packs (g) |

Lyophilized Granules prepared from liquid composition composed of:

| | | |
|---|---|---|
| 1 | Sildenafil Citrate | 140.48 |
| 2 | Sodium hydroxide | 24.60 |
| 3 | Mannitol | 230.92 |
| 4 | HPC-L | 4.0 |
| 5 | Purified Water | q.s to 1200 g |

Blend Composition

| | | |
|---|---|---|
| 6 | Sorbitol | 543.50 |
| 7 | Orange flavor | 3.00 |
| 8 | Neotame | 0.50 |
| 9 | Tartaric acid pellets | 50.00 |
| 10 | Liquid paraffin | 3.00 |

Example 2

| | |
|---|---|
| | Quantity per 1000 |
| Ingredients | Stick Packs (g) |

Lyophilized Granules prepared from liquid composition composed of:

| | | |
|---|---|---|
| 1 | Sildenafil Citrate | 140.48 |
| 2 | Sodium hydroxide | 20.00 |
| 3 | Mannitol | 230.52 |
| 4 | HPC-L | 4.0 |
| 5 | Purified Water | q.s to 1200 g |

Blend Composition

| | | |
|---|---|---|
| 6 | Sorbitol | 548.50 |
| 7 | Orange flavor | 3.00 |
| 8 | Neotame | 0.50 |
| 9 | Tartaric acid pellets | 50.00 |
| 10 | Liquid paraffin | 3.00 |

Example 3

| | |
|---|---|
| | Quantity per 1000 |
| Ingredients | Stick Packs (g) |

Lyophilized Granules prepared from liquid composition composed of:

| | | |
|---|---|---|
| 1 | Sildenafil Citrate | 140.48 |
| 2 | Sodium hydroxide | 30.12 |
| 3 | Mannitol | 230.52 |
| 4 | HPC-L | 4.0 |
| 5 | Purified Water | q.s to 1200 g |

Blend Composition

| | | |
|---|---|---|
| 6 | Sorbitol | 538.38 |
| 7 | Orange flavor | 3.00 |
| 8 | Neotame | 0.50 |
| 9 | Tartaric acid pellets | 50.00 |
| 10 | Liquid paraffin | 3.00 |

Example 4

| | |
|---|---|
| | Quantity per 1000 |
| Ingredients | Stick Packs (g) |

Lyophilized Granules prepared from liquid composition composed of:

| | | |
|---|---|---|
| 1 | Sildenafil Citrate | 140.48 |
| 2 | Sodium hydroxide | 24.60 |
| 3 | Mannitol | 230.92 |
| 4 | HPC-L | 4.0 |
| 5 | Purified Water | q.s to 1200 g |

Blend Composition

| | | |
|---|---|---|
| 6 | Sorbitol | 543.50 |
| 7 | Orange flavor | 3.00 |
| 8 | Neotame | 0.50 |
| 9 | Citric acid | 50.0 |
| 10 | Liquid paraffin | 3.00 |

### Manufacturing Procedure

### I. Lyophilized Granules

1. Sodium hydroxide was dissolved in 370 mL of purified water under stirring on overhead stirrer.
2. Sildenafil citrate was dispersed uniformly in the solution of step 1 under stirring.
3. The dispersion obtained in step 2 was homogenized using homogenizer and overhead stirrer.
4. Hydroxypropyl cellulose L was dissolved in water (166.5 g) and the solution was added to dispersion of step 2 under stirring.
5. Mannitol was added to the dispersion.
6. The final weight of the solution/dispersion was made to 1200 g using purified water.
7. Mixing of the solution/dispersion was continued using a magnetic/overhead stirrer.
8. This solution/dispersion was filled in stainless steel tray and freeze-dried in a lyophilizer.
9. After complete freeze drying, the lyophilized cake was sieved through a #40 sieve to obtain the lyophilized granules.

### II. Blend Composition

10. Sorbitol was passed through a #40 sieve and neotame with orange flavor were passed through a 60 #sieve. Sieved sorbitol, neotame and orange flavor were mixed in a rapid mixer granulator
11. Liquid paraffin was added dropwise to the mixture (80 g) from step-10 and mixed to obtain a uniformly distributed composition.
12. The composition from step 11 was sieved through a #40 sieve and mixed with mixture from step 10 using a rapid mixture granulator
13. The mixture obtained in step 12 was passed through a 40 #sieve, and transferred to a double cone blender
14. Organic acid was added to the blender of step 13 and the mixture was blended to obtain the blend composition

### III. Formulation

15. The lyophilized granules from step 9 were mixed with the blend composition from step 14 in a double cone blender to obtain the free flowing Sildenafil formulation.
16. The free flowing formulation was filled into stick packs and sealed.

Comparative pharmaceutical formulations were prepared essentially as described in Example 1 with the following changes:

Comparative Example A

| | |
|---|---|
| | Quantity per 1000 |
| Ingredients | Stick Packs (g) |

Lyophilized Granules prepared from liquid composition composed of:

| | | |
|---|---|---|
| 1 | Sildenafil Citrate | 140.48 |
| 2 | Sodium hydroxide | 24.60 |
| 3 | Mannitol | 230.92 |
| 4 | HPC-L | 4.0 |
| 5 | Purified Water | q.s to 1200 g |

Blend Composition

| | | |
|---|---|---|
| 6 | Sorbitol | 593.50 |
| 7 | Orange flavor | 3.00 |
| 8 | Neotame | 0.50 |
| 9 | Tartaric acid pellets | 0.00 |
| 10 | Liquid paraffin | 3.00 |

Comparative Example B

| | |
|---|---|
| | Quantity per 1000 |
| Ingredients | Stick Packs (g) |

Lyophilized Granules prepared from liquid composition composed of:

| | | |
|---|---|---|
| 1 | Sildenafil Citrate | 140.48 |
| 2 | Sodium hydroxide | 30.12 |
| 3 | Mannitol | 149.45 |
| 4 | HPC-L | 4.0 |
| 5 | Purified Water | q.s to 1000 g |

Blend Composition

| | | |
|---|---|---|
| 6 | Sorbitol | 644.66 |
| 7 | Orange flavor | 3.00 |
| 8 | Neotame | 0.50 |
| 9 | Tartaric acid pellets | 24.79 |
| 10 | Liquid paraffin | 3.00 |

Comparative Example C

| | |
|---|---|
| Ingredients | Quantity per 1000 Stick Packs (g) |

Lyophilized Granules prepared from liquid composition composed of:

| | | |
|---|---|---|
| 1 | Sildenafil Citrate | 140.48 |
| 2 | Sodium hydroxide | 30.12 |
| 3 | Mannitol | 149.45 |
| 4 | HPC-L | 4.0 |
| 5 | Purified Water | q.s to 1000 g |

Blend Composition

| | | |
|---|---|---|
| 6 | Sorbitol | 636.5 |
| 7 | Orange flavor | 3.00 |
| 8 | Neotame | 0.50 |
| 9 | Tartaric acid pellets | 33.0 |
| 10 | Liquid paraffin | 3.00 |

Comparative Example D

| | |
|---|---|
| Ingredients | Quantity per 1000 Stick Packs (g) |

Lyophilized Granules prepared from liquid composition composed of:

| | | |
|---|---|---|
| 1 | Sildenafil Citrate | 140.48 |
| 2 | Sodium hydroxide | 30.12 |
| 3 | Mannitol | 149.45 |
| 4 | HPC-L | 4.0 |
| 5 | Purified Water | q.s to 1000 g |

Blend Composition

| | | |
|---|---|---|
| 6 | Sorbitol | 628.05 |
| 7 | Orange flavor | 3.00 |
| 8 | Neotame | 0.50 |
| 9 | Tartaric acid pellets | 41.40 |
| 10 | Liquid paraffin | 3.00 |

### In-Vitro Dissolution

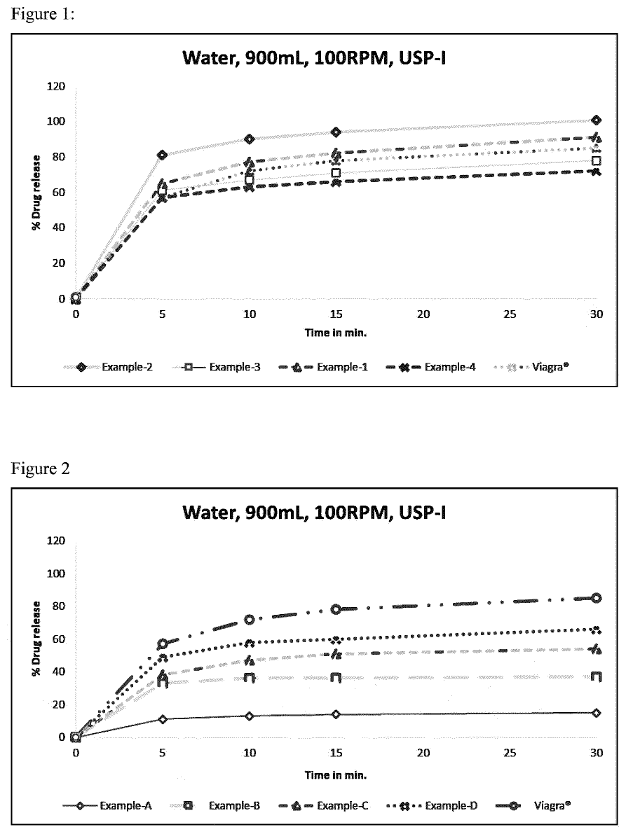

In vitro dissolution testing was performed in accordance with USP Type I, basket (100 #) at 100 rpm in 900 mL of water as dissolution medium, maintained at 37° C. #0.5° C. The release profiles of Examples 1 to 4 in comparison to the Viagra® 100 mg tablet are shown in FIG. 1. The release profiles of Comparative Examples A to D in comparison to Viagra® 100 mg tablet are shown in FIG. 2. Formulations according to Examples 1 to 4 demonstrated similar or faster release at 5 minutes as compared to Viagra® 100 mg tablet.

### Bioavailability of the Stable Oral Composition

A 2-way, randomized, crossover study was carried out to compare the bioavailability of Example 1 at a dose of 100 mg versus Viagra® (sildenafil citrate) 100 mg film-coated tablet in 48 healthy adult males under fasting conditions.

In each study period, a single dose of sildenafil citrate composition was administered orally, in the morning, following a 10-hour overnight fast. Fasting condition was maintained up to 4 hours following dosing. The administration was done as follows:

Example 1 containing 100 mg of sildenafil citrate, administered orally to the volunteers without water.

An oral dose of 100 mg Viagra® tablet was administered with about 240 mL of water.

Sildenafil plasma concentrations were quantified through serial blood sampling and sildenafil pharmacokinetic (PK) parameters determined by non-compartmental analysis. Parameters of interest were, peak plasma concentration (Cmax), area under the plasma concentration curve (AUC), time to peak plasma concentration (Tmax) and time lag until observed sildenafil concentration (Tlag). The AUCs were measured through linear trapezoidal method.

Results

| Parameters | Example 1 (100 mg) | Viagra ® tablet 100 mg |
|---|---|---|
| Tlag (min) | 0-12 | 3-20 |
| Tmax (h) | 0.33-2.33 | 0.33-2.67 |
| Cmax (ng/ml) | 378-1950 | 359-1996 |
| AUC0-10 min | 3.51 | 0.29 |
| (ng · h/mL)* | (0-46) | (0-2) |
| AUC0-30 min | 170 | 105 |
| (ng · h/mL)* | (3-606) | (3-360) |
| AUC0-t (ng · h/mL) | 1496-6002 | 1314-6825 |
| AUC0-inf (ng · h/mL) | 1518-6436 | 1333-7099 |

*observed range of PK parameter is given in parentheses

### Evaluation 1: Bulk Density & Tapped Density Results

Bulk and tapped density are important to understand flow properties of formulations. The bulk density of a material is the ratio of the mass to the volume (including the interparticulate void volume) of an untapped powder sample. The tapped density is an increased bulk density attained after mechanically tapping a container containing the powder sample. The tapped density is obtained by mechanically tapping a graduated cylinder containing the sample until little further volume change is observed. Because the interparticulate interactions influencing the bulking properties of a powder are also the interactions that interfere with powder flow, a comparison of the bulk and tapped densities can give a measure of the relative importance of these interactions in a given powder. Such a comparison is often used as an index of the ability of the powder to flow, for example the Compressibility Index or the Hausner Ratio:

### Compressibility Index (Carr Index):

$$\frac{100 ( {V}_{0} - {V}_{f} )}{{V}_{0}}$$

### Hausner Ratio:

$$\frac{{V}_{0}}{{V}_{f}}$$

V₀=unsettled apparent volume (bulk volume) V_{f}=final tapped volume

The lower the values of Compressibility Index and Hausner Ratio, the better the flow properties of the powder.

### Procedure for Determining Bulk Density:

According to US Pharmacopoeia (USP) Chapter <616>, Method I, using a 100-mL cylinder readable to 1 mL and an amount of powder such that its untapped apparent volume is between 50 and 100 mL.

### Procedure for Determining Tapped Density:

According to US Pharmacopoeia (USP) Chapter <616>, Method I, with the following details:
A 100-mL graduated cylinder (readable to 1 mL) weighing 130±16 g is mounted on a holder weighing 240±12 g.
10, 500, and 1250 taps on the powder sample are conducted and corresponding volumes V₁₀, V₅₀₀, and V₁₂₅₀ to the nearest graduated unit are measured.
If the difference between V₅₀₀ and V₁₂₅₀ is less than or equal to 1 mL, V₁₂₅₀ is the tapped volume.
If the difference between V₅₀₀ and V₁₂₅₀ exceeds 1 mL, the measurement is repeated in increments such as 1250 taps, until the difference between succeeding measurements is less than or equal to 1 mL.

Results

| | Bulk Density | Tapped Density | Carr | Hausner |
|---|---|---|---|---|
| Formulations | (g/ml) | (g/ml) | Index | Ratio |
| Example 1 | 0.47 | 0.61 | 23.26 | 1.30 |
| Example 2 | 0.44 | 0.59 | 26.09 | 1.35 |
| Example 3 | 0.44 | 0.63 | 28.89 | 1.41 |
| Example 4 | 0.45 | 0.59 | 22.73 | 1.29 |
| Comparative Example A | 0.45 | 0.59 | 22.73 | 1.29 |
| Comparative Example B | 0.44 | 0.61 | 26.67 | 1.36 |
| Comparative Example C | 0.44 | 0.61 | 26.67 | 1.36 |
| Comparative Example D | 0.44 | 0.61 | 26.67 | 1.36 |

### Evaluation 2: Angle of Repose:

The angle of repose has been used to characterize the flow properties of solids. Angle of repose is a characteristic related to interparticulate friction or resistance to movement between particles. The angle of repose is a constant, three-dimensional angle (relative to the horizontal base) assumed by a cone-like pile of material.

### Procedure (as Per USP General Chapter <1174>):

The angle of repose is formed on a fixed base with a retaining lip to retain a layer of powder on the base. The base should be free of vibration. The height of the funnel is varied to carefully build up a symmetrical cone of powder. Care should be taken to prevent vibration as the funnel is moved. The funnel height should be maintained approximately 2-4 cm from the top of the powder pile as it is being formed in order to minimize the impact of falling powder on the tip of the cone. If a symmetrical cone of powder cannot be successfully or reproducibly prepared, this method is not appropriate. The angle of repose is determined by measuring the height of the cone of powder and calculating the angle of repose (a) from the following equation:

tan (a)=height/0.5 base.

Results

| Formulations | Angle of repose (degree) | Flow behavior |
|---|---|---|
| Example 1 | 32 | Good |
| Example 2 | 36 | Fair |
| Example 3 | 38 | Fair |
| Example 4 | 31 | Good |
| Comparative Example A | 38 | Fair |
| Comparative Example B | 37 | Fair |
| Comparative Example C | 37 | Fair |
| Comparative Example D | 38 | Fair |

### Evaluation 3: Moisture Content:

The pharmaceutical formulations were placed in open petri dishes, which were then placed in a Climacel maintained at 25° C./75% RH for simulating exposure to high humidity. The moisture content of each formulation was analyzed after 1, 2 and 6 hours in the Climacel. Moisture content was evaluated by using USP General Chapter 921-Water Determination method.

Results

| | Moisture Content | | | |
|---|---|---|---|---|
| | (wt %) | | | |
| Formulations | Initial | 1 hour | 2 hours | 6 hours |
| Example 1 | 1.70 | 5.50 | 5.27 | 8.26 |
| Example 2 | 1.26 | 2.94 | 3.49 | 6.02 |
| Example 3 | 1.91 | 5.05 | 8.17 | 15.07 |
| Example 4 | 1.65 | 4.90 | 7.20 | 14.66 |
| Comparative Example A | 1.49 | 4.71 | 7.55 | 16.13 |
| Comparative Example B | 1.89 | 5.34 | 8.62 | 10.77 |
| Comparative Example C | 2.04 | 4.94 | 6.42 | 9.63 |
| Comparative Example D | 1.86 | 4.90 | 7.99 | 9.22 |

### Evaluation 4: Anti-Sticking Properties

The anti-sticking properties of the formulation was measured by determining the residual content using the following procedure:

### Procedure:

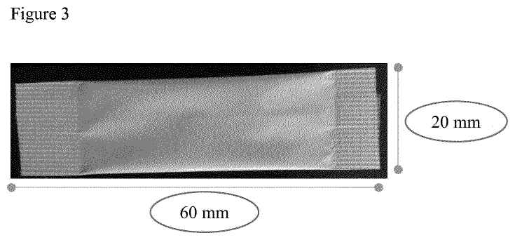

The pharmaceutical formulations were filled uniformly in 10 stick packs made from flexible laminates as depicted in FIG. 3. The fill weight of each stick pack was noted down for further reference. Filled stick packs were sealed using a sealing machine. The sealed stick packs were kept on vibratory shifter for 30 minutes for entrapping the formulations into the corners of the stick pack. Each stick pack was opened by cutting from one sealed side. The formulation from each stick pack was removed and weighed on a calibrated balance to measure the amount of formulation recovered from each stick pack. The residual content was calculated using the following formula:

$$( Fill weight of stick pack ) - ( Content recovered from stick pack ) = Residual content$$

Results

| Formulations | % Residual content | Standard Deviation |
|---|---|---|
| Example 1 | 2.28 | 0.24 |
| Example 2 | 2.55 | 0.87 |
| Example 3 | 1.73 | 0.64 |
| Example 4 | 2.59 | 0.49 |
| Comparative Example A | 2.78 | 0.50 |
| Comparative Example B | 1.89 | 0.91 |
| Comparative Example C | 2.21 | 0.74 |
| Comparative Example D | 2.24 | 0.47 |

1. A solid pharmaceutical formulation comprising a mixture of (1) lyophilized granules comprising sildenafil or a pharmaceutically acceptable salt thereof as active ingredient and (2) a non-lyophilized composition, wherein the formulation comprises 35% to 50% by weight of the lyophilized granules (1) and dissolves in the oral cavity and the non-lyophilized composition (2) is a solid blend composition comprising flow enhancing agents.

2. The solid pharmaceutical formulation according to claim 1, wherein the formulation provides at least one of the following pharmacokinetic parameters upon oral administration to a human subject: AUC (0-10 min) of at least 3.5 ng·h/mL; and AUC (0-30 min) of at least 150 ng·h/mL, wherein AUC refers to area under a sildenafil plasma concentration versus time curve.

3. The solid pharmaceutical formulation according to claim 1, wherein the flow enhancing agents are a combination of sorbitol and liquid paraffin.

4. The solid pharmaceutical formulation according to claim 3, wherein:
the formulation comprises 50% to 65% by weight of the solid blend composition (2) and further comprises 0% to 15% by weight of one or more pharmaceutically acceptable excipients as further constituent components of the formulation,
the solid blend composition (2) comprises 85% to 93% by weight of sorbitol, 5% to 14.9% by weight of an organic acid selected from one or both of tartaric acid and citric acid, and 0.1% to 5% by weight of liquid paraffin, and
the active ingredient constitutes 0.01% to 30% by weight of the formulation.

5. The solid pharmaceutical formulation according to claim 4, wherein the solid blend composition (2) comprises 90.5% to 92.0% by weight of sorbitol, 5.0% to 9.0% by weight of an organic acid selected from one or both of tartaric acid and citric acid, and 0.5% to 3.5% by weight of liquid paraffin.

6. The solid pharmaceutical formulation according to claim 4, wherein the formulation comprises 37.5% to 42.5% by weight of the lyophilized granules (1), 57.5% to 62.5% by weight of the solid blend composition (2), and 0.0% to 5.0% by weight of the one or more pharmaceutically acceptable excipients as further constituent components of the formulation, and the active ingredient constitutes 10% to 20% by weight of the formulation.

7. The solid pharmaceutical formulation according to claim 6, wherein the formulation consists of 37.5% to 42.5% by weight of the lyophilized granules (1) and 57.5% to 62.5% by weight of the solid blend composition (2), wherein the solid blend composition (2) consists of 90.5% to 92.0% by weight of sorbitol, 5.0% to 9.0% by weight of an organic acid selected from one or both of tartaric acid and citric acid, and 0.5% to 3.5% by weight of liquid paraffin.

8. The solid pharmaceutical formulation according to claim 1, wherein the active ingredient is sildenafil citrate.

9. The solid pharmaceutical formulation according to claim 4, wherein the organic acid is tartaric acid.

10. The solid pharmaceutical formulation according to claim 1, wherein the formulation is a free-flowing formulation.

11. The solid pharmaceutical formulation according to claim 1, wherein the lyophilized granules (1) comprise a base.

12. The solid pharmaceutical formulation according to claim 1, wherein the lyophilized granules (1) comprise 35% to 46% by weight of sildenafil or a pharmaceutically acceptable salt thereof, 39% to 65% by weight of sugar alcohol, and 0% to 15% by weight of base.

13. The solid pharmaceutical formulation according to claim 11, wherein the weight ratio of organic acid in the solid blend composition (2) to the base in the lyophilized granules (1) is at least 1.6:1.

14. A package containing the formulation according to claim 1.

15. The package according to claim 14, wherein the package is selected from the group consisting of a sachet and a stick pack.

16. A process for manufacturing a pharmaceutical formulation according to claim 1, comprising preparing a solid blend composition comprising sorbitol and liquid paraffin.

17. A process for manufacturing a pharmaceutical formulation according to claim 1, comprising:
(a) forming lyophilized granules (1) by subliming solvent from a preparation comprising sildenafil or a pharmaceutically acceptable salt thereof and solvent;
(b) forming a solid blend composition (2) comprising or consisting of sorbitol, liquid paraffin, and an organic acid selected from one or both of tartaric acid and citric acid; and
(c) mixing the lyophilized granules (1) obtained in step (a) with the solid blend composition (2) obtained in step (b).

18. The solid pharmaceutical formulation according to claim 11 wherein the base comprises sodium hydroxide.

19. The solid pharmaceutical formulation according to claim 13, wherein the weight ratio of organic acid in the solid blend composition (2) to the base in the lyophilized granules (1) is at least 2:1.
